(19) United States
(12) Patent Application Publication
(10) Pub. No.: US 2023/0062882 A1
(43) Pub. Date: Mar. 2, 2023

(54) Germicidal Donnable Hand Cloth

(71) Applicant: Western Technology, Inc., Bremerton, WA (US)

(72) Inventor: Michael Kralik, Bremerton, WA (US)

(21) Appl. No.: 17/463,999

(22) Filed: Sep. 1, 2021

(51) Int. Cl. A61L 2/10; A41D 19/00

(52) U.S. Cl. CPC A61L 2/10; A41D 19/0024; A41D 2400/26; A61L 2202/11; A61L 2202/14; A61L 2202/16

(57) ABSTRACT

A germicidal hand cloth can comprise a fabric substrate operable to receive a hand. The fabric substrate can define an interior and an exterior opposite the glove interior. Additionally, the germicidal hand cloth can include a light source supported by the covering. The light source can be operable to emit ultraviolet C spectrum (UVC) light having a wavelength from about 200 nm to about 280 nm. The UVC light can be directed toward the exterior to kill germs located exterior to and remote from the fabric substrate.

### BACKGROUND

Most current surface disinfection methods use chemical agents. Chemical disinfection can be confusing and inconsistent because of the many products available. To ensure proper disinfection, the selected agent, manner of use (e.g., immersion, contact, etc.), and the treatment time must be correctly and consistently applied. Ultraviolet (UV) light produced by mercury lamps has been used for over 100 years and proven effective in disinfection protocols. Despite this history, UV light is not currently prominently used for surface disinfection.

### SUMMARY

A germicidal hand cloth can comprise a fabric substrate operable to receive a hand. The fabric substrate can define an interior and an exterior opposite the glove interior. Additionally, the germicidal hand cloth can include a light source supported by the covering. The light source can be operable to emit ultraviolet C spectrum (UVC) light having a wavelength from about 200 nm to about 280 nm. The UVC light can be directed toward the exterior to kill germs located exterior to and remote from the fabric substrate.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1A is a front view of a germicidal glove in accordance with an example of the present disclosure.

FIG. 1B is a back view of the germicidal glove of FIG. 1A in accordance with an example of the present disclosure.

FIG. 2 illustrates light emission modes from an optical fiber.

FIG. 3 is a front view of a germicidal glove in accordance with an example of the present disclosure.

FIG. 4 illustrates integrating an optical fiber with a textile fabric by weaving, in accordance with several examples.

FIG. 5 illustrates integrating an optical fiber with a textile fabric by embroidering, in accordance with several examples.

FIG. 6 illustrates integrating an optical fiber with a textile fabric by knitting, in accordance with several examples.

FIG. 7 is a detailed cross-sectional view of the germicidal glove of FIGS. 1A and 1B, in accordance with an example of the present disclosure.

FIG. 8 is a front view of a germicidal hand cloth in accordance with another example.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The term “glove” as used herein is meant to include any suitable form factor for covering all or a portion of a human hand, such as a typical glove form factor where each finger or digit is separately covered, a mitten form factor where multiple fingers or digits are covered together (e.g. mittens), or any combination of form factors or configurations. Additionally, a “glove” as disclosed herein can be configured to cover more than a user’s hand (e.g., extend to cover all or a portion of an arm) and/or leave a portion of a hand exposed (e.g., an exposed back of the hand and/or a “fingerless” glove configuration).

The singular forms “a,” “an,” and “the” include plural referents unless the context clearly dictates otherwise. Thus, for example, reference to “an optical fiber” includes reference to one or more of such features.

As used herein, “donnable” refers to a capacity to be worn or retained on the hand.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C“explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Germicidal Glove

A germicidal hand cloth can comprise a fabric substrate operable to receive a hand. The fabric substrate can define an interior and an exterior opposite the glove interior. Additionally, the germicidal hand cloth can include a light source supported by the covering. The light source can be operable to emit ultraviolet C spectrum (UVC) light having a wavelength from about 200 nm to about 280 nm. The UVC light can be directed toward the exterior to kill germs located exterior to and remote from the fabric substrate.

Generally, the fabric substrate can be formed of woven textile, non-woven fabric, or the like. The thickness of the fabric substrate can vary depending on desired flexibility and thermal insulation; however, thicknesses from about 0.10 mm to 2 mm, and often from 0.15 mm to 1 mm. Further, the fabric substrate can be a flexible material formed as a glove or as a planar sheet.

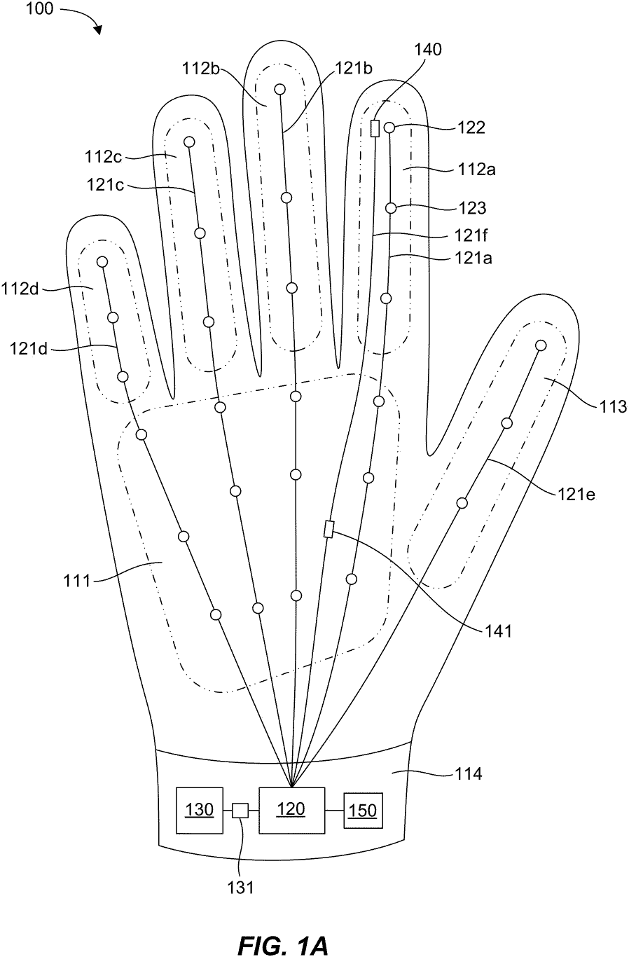

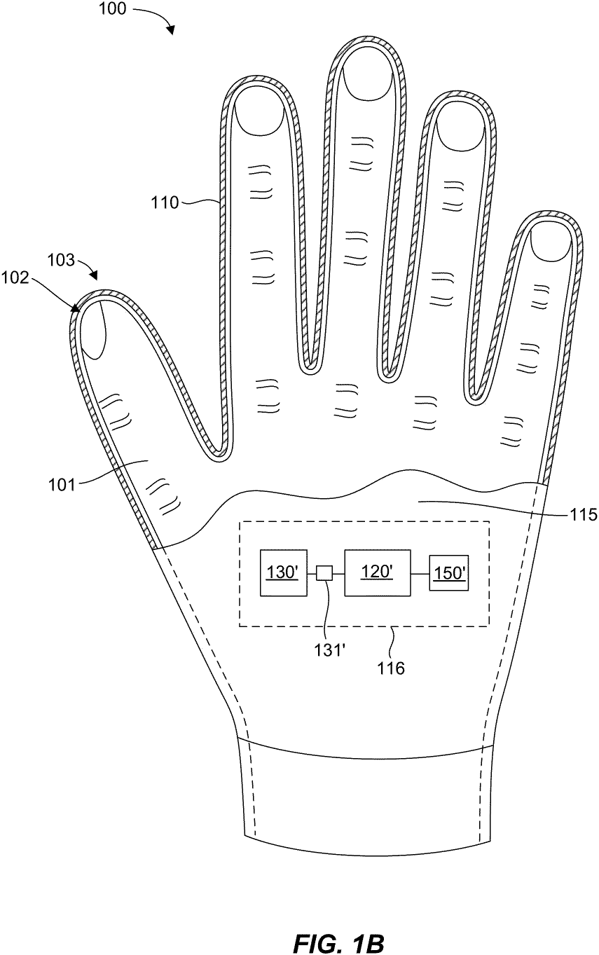

With reference to FIGS. 1A and 1B, a germicidal glove 100 is illustrated in accordance with an example of the present disclosure. The germicidal glove 100 can include a covering 110 for a hand 101 (FIG. 1B). The covering 110 can define a glove interior 102 operable to receive the hand 101. The covering 110 can also define a glove exterior 103 opposite the glove interior 102. In one aspect, the covering 110 can include at least one of a palm portion 111, a finger portion 112a-d, or a thumb portion 113. The covering 110 can include other portions, as desired, to provide any suitable structural and/or covering benefit, such as a wrist portion 114 and/or a back portion 115.

The germicidal glove 100 can include a light source 120 supported by the covering 110. The germicidal glove 100 can also include a power source 130 supported by the covering 110 and operably coupled to the light source 120. Any suitable type of power source can be utilized, such as a battery (e.g., a rechargeable battery). A suitable power switch 131 can be included to enable the user to control power from the power source 130 to the light source 120, such as by providing a user interface (e.g., a button, tab, lever, etc.). In some examples, the germicidal glove 100 can include one or more sensors 140, 141 supported about the covering 110 to facilitate control of the light source 120 (e.g., turning on/off), as described in more detail below. In this case, the germicidal glove 100 can include a control system 150 operably coupled to the sensors 140, 141 to monitor the sensors 140, 141 and control various functional and operational aspects of the light source 120, such as receiving sensor inputs and controlling the light source based on the sensor inputs. The control system 150 can include any suitable hardware (e.g., a processor, memory, etc.) and/or software that can facilitate monitoring the sensors 140, 141 and controlling the light source 120.

The light source 120, the power source 130, the power switch 131, and the control system 150 can be supported about the covering 110 in any suitable location and in any suitable manner. For example, FIG. 1A illustrates the light source 120, the power source 130, the power switch 131, and the control system 150 supported about the wrist portion 114. Alternatively, as illustrated in FIG. 1B, a light source 120', a power source 130', a power switch 131', and a control system 150' can be supported about the back portion 115. The locations for these components can be selected based on user comfort, accessibility, packaging, unobtrusiveness, etc., and the components can be located in the same location (e.g., on the wrist portion 114 or the back portion 115) or in various different locations about the glove 100. In one aspect, the covering 110 can comprise a pocket 116 operable to receive and contain one or more of the light source 120', the power source 130', the power switch 131', or the control system 150'.

The light source 120 can be operable to emit ultraviolet C spectrum (UVC) light. UVC light has a wavelength from about 200 nm to about 280 nm. In one aspect, the UVC light emitted from the light source 120 can have a wavelength from about 260 nm to about 270 nm, for example, as a spectral range of peak power output for optimal germicidal effect across a range of pathogens. The germicidal effects of UVC irradiation results in cellular damage by photohydration, photosplitting, photodimerization, and photocrosslinking, thereby inhibiting cellular replication. In one aspect, the light source 120 can comprise one or more light emitting diodes (LEDs), as commonly known in the art, which can provide efficient light generation and low power consumption.

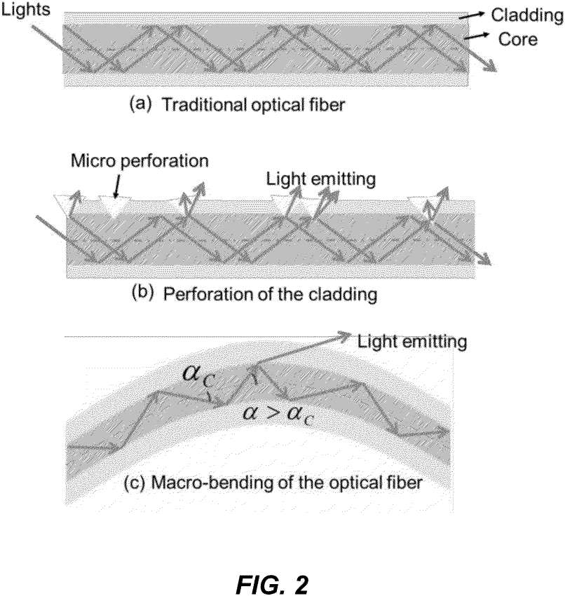

The UVC light can be directed outwardly from the glove exterior 103 from one or more locations about the covering 110. For example, the UVC light can be directed from at least one of the palm portion 111, the finger portion 112a-d, or the thumb portion 113. In one aspect, UVC light can be projected (e.g., uniformly) across the palm portion 111, the finger portion 112a-d, and/or the thumb portion 113. For example, as illustrated in FIG. 1A, the germicidal glove 100 can include one or more optical fibers 121a-e operably coupled to the light source 120 to direct the UVC light from the palm portion 111, the finger portion 112a-d, and/or the thumb portion 113 via an end 122 of an optical fiber, an opening or perforation 123 in an optical fiber, and/or via macro-bending of an optical fiber (see FIG. 2), as known in the art. In another example, all or a portion of an optical fiber 121a-e (e.g., within the palm portion 111, the finger portion 112a-d, and/or the thumb portion 113) can be configured to illuminate and emit the UVC light. In such cases, a single (e.g., remote) light source (e.g., comprising one or more LEDs) can supply UVC light for all emitting locations about the covering 110.

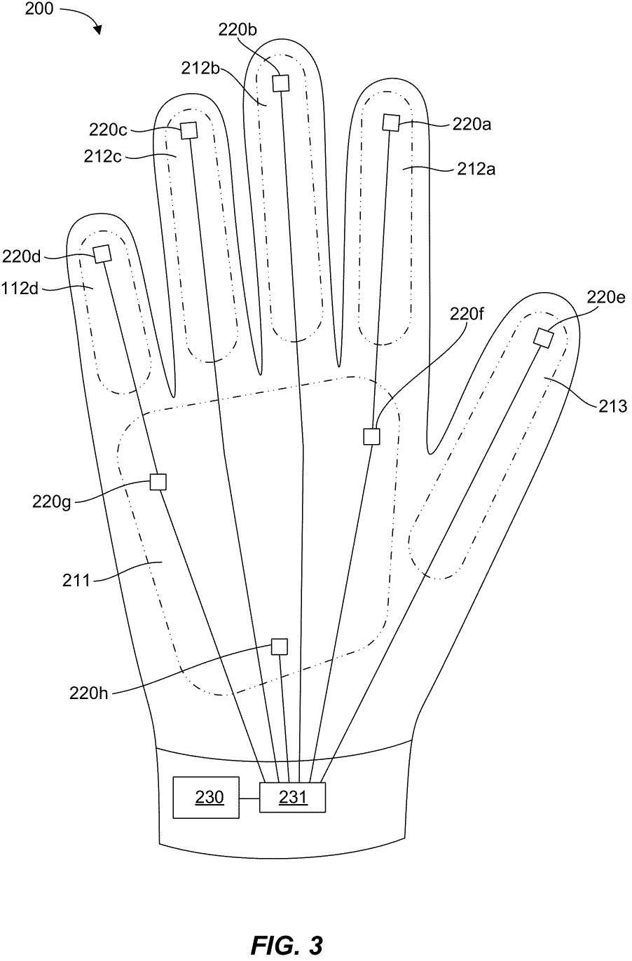

Alternatively, as illustrated in FIG. 3, a germicidal glove 200 can include one or more light sources 220a-h located on a palm portion 211, a finger portion 212a-d, and/or a thumb portion 213 to direct UVC light from the palm portion 211, the finger portion 212a-d, and/or the thumb portion 213. In this case, the UVC light can be generated and emitted from the same location (e.g., from the palm portion 211, the finger portion 212a-d, and/or the thumb portion 213). A power source 230 and associated power switch 231 can be operably connected to the light sources 220a-h to control operation of the light sources 220a-h. In one aspect, one or more sensors and a control system (not shown) can be included to control operation of the light sources 220a-h in a similar manner as discussed herein regarding the germicidal glove 100.

Referring again to FIGS. 1A and 1B, the germicidal glove 100 can be a short-range disinfection system where the UVC light can be directed toward the glove exterior 103 to kill germs located exterior to the covering 110, typically on a surface that may be in close proximity to the germicidal glove 100 (e.g., within about 2.5 cm). In some examples, the intensity of UVC light emitted from the glove 100 can be operable to have a germicidal effect up to about 2.5 cm of the germicidal glove 100. In particular examples, the intensity of UVC light emitted from the germicidal glove 100 can be operable to have a germicidal effect up to about 1 cm from the germicidal glove 100.

The sensors 140, 141 can be or include any suitable type of sensor, such as a contact sensor, a pressure sensor, a tactile sensor, a proximity sensor, an impact sensor, etc. to control the light source 120 (e.g., turn on/off) when the germicidal glove 100 is in position relative to a surface to be sanitized and/or removed from such a surface. Suitable types of sensors can include a capacitive sensor, a flex sensor, an optical sensor, an electro-optical sensor, a photoelectric sensor, an accelerometer, a strain gauge, a piezoelectric sensor, etc., or any other suitable type of sensor known in the art that can be used to control operation of the light source 120 consistent with the manner described herein.

As discussed above, the sensor 140, 141 can be located on at least one of the palm portion 111, the finger portion 112a-d, or the thumb portion 113. In one aspect, one or more of the sensors 140, 141 can be operable to detect contact or pressure on the glove exterior 103 and/or the proximity of a surface within a given distance from the sensor 140, 141 (e.g., less than about 2.5 cm or less than about 1 cm). When contact, pressure, or proximity is detected, the light source 120 can emit the UVC light to disinfect the surface. On the other hand, when contact, pressure, or proximity is not detected by the sensor 140, 141 (or if a face or skin is recognized by one or more of the sensors 140, 141 using any suitable technique known in the art), the light source 120 can be automatically turned off to avoid harming skin, eyes, etc. with UVC light. In some examples, the sensor 140, 141 can be associated with one or more locations of the covering 110 (e.g., the palm portion 111, the finger portion 112a-d, and/or the thumb portion 113), and can be operable to control UVC light emissions from the associated location(s) of the covering 110. For example, the sensor 140 can be associated with the finger portion 112a and the sensor 141 can be associated with the palm portion 111. Other sensors can likewise be associated with the other finger portions 112b-d and the thumb portion 113. Such a sensor arrangement can provide localized control over the emission of UVC light, which can reduce the risk of scattered radiation and inadvertent harm caused by the UVC light. It should be recognized that any suitable number of sensors can be utilized in any location and for any purpose in accordance with the principles disclosed herein

As discussed above, the sensors 140, 141 can be optical sensors. In one aspect, one or more of the sensors 140, 141 can comprise an optical wavelength filter, a wavelength-specific reflector, and/or any other suitable optical sensor technology. For example, one or more of the sensors 140, 141 can comprise a strain gauge, a thermal sensor, an accelerometer, and/or any other suitable sensor type for sensing a condition relative to the contact, pressure, or proximity of a surface to be sanitized by the germicidal glove 100. In one aspect, the sensors 140, 141 can be configured for suitable sensitivity, precision, and accuracy in monitoring the contact, pressure, or proximity of a surface to be sanitized by the germicidal glove 100. Optical sensors are advantageously utilized as sensors for this purpose due to optical sensors being insensitive to electromagnetic interference (EMI) and electrostatic discharge (ESD), while being rugged, extremely small, and lightweight.

In one aspect, the germicidal glove 100 can include an optical fiber 121f (FIG. 1A), which can be coupled to or include one or more of the sensors 140, 141. For example, one or more of the sensors 140, 141 can be integrally formed with the optical fiber 121f (e.g., an optical wavelength filter and/or a wavelength-specific reflector can be formed (e.g., inscribed or “written”) on an internal wall of the optical fiber 121f). Although the sensors 140, 141 are shown on an optical fiber 121f that does not emit UVC light, in some examples, the optical fiber 121f can be configured to emit UVC light or the sensors 140, 141 can be included in an optical fiber (e.g., one or more of the optical fibers 121a-e) that is configured to emit UVC light. The optical fibers 121a-f can be of any suitable size or construction. In one example, one or more of the optical fibers 121a-f can be micron scale in size (e.g., 150-250 µm in diameter). Similarly, the sensors 140, 141 can be micron scale in size (e.g., 5-10 µm). In one aspect, the optical fibers 121a-f can be a polymer, bend-insensitive fiber for optimum performance in any prospective environment.

In one example, one or more of the sensors 140, 141 can comprise a fiber Bragg grating (FBG), which forms the sensing element. FBGs and their operation and usefulness as optical sensors is well-known in the art. FBGs are micron-scale, wavelength-selective mirrors that reflect a single, specific wavelength (referred to as the Bragg wavelength) and allow the rest of the optical signal to pass through. When the optical fiber 121f, and therefore the FBG, is deformed (e.g., stretched, compressed, or undergoes thermal expansion and contraction), the Bragg or reflected wavelength changes. A suitable demodulation technique can be employed to observe the change in wavelength and translate this into a measurement of a desired sensed condition (e.g., strain, temperature, acceleration, etc.). In another example, one or more of the sensors 140, 141 can comprise a Fabry-Perot sensor, which is also well-known in the art as an optical sensor.

In another example, one or more of the sensors 140, 141 can comprise a fiber optic micro bend sensor as known in the art. Microbending loss is a type of light intensity loss caused by defects and small geometrical perturbations along the fiber axis, the deformation of which is in the order of micrometers. Light propagating in the microbending optical fiber with a given intensity can be modulated by external load signals such as strain, pressure, and acceleration resulting in a varied light intensity. Therefore, an output intensity can be obtained to monitor target physical parameters such as contact or pressure.

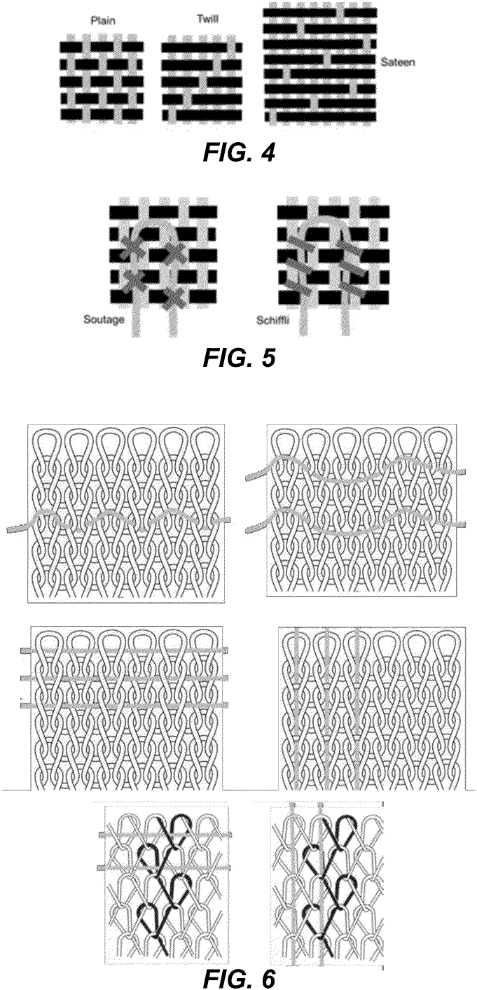

In one aspect, one or more of the optical fibers 121a-f can be integrated with or embedded into a textile fabric. For example, one or more of the optical fibers 121a-f can be threaded or woven into a textile or fabric. In some examples, the diameter of the optical fibers 121a-f can be similar to the thickest threads used in the fabric. One or more of the optical fibers 121a-f can be integrated with a textile fabric in any suitable manner, such as weaving (e.g., plain, twill, and sateen fabrics as shown in FIG. 4), embroidering (e.g., soutage and schiffli techniques as shown in FIG. 5), knitting (e.g., as shown in FIG. 6), or any other suitable textile fabrication technique, into single or multilayer structures.

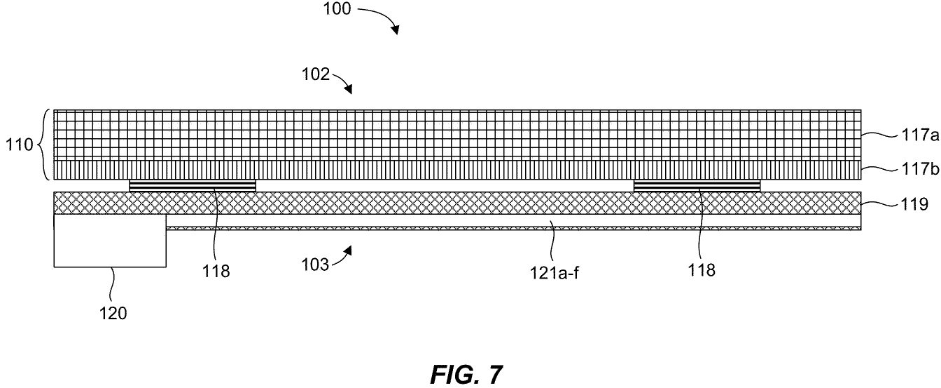

With further reference to FIGS. 1A and 1B, in one aspect, as illustrated in FIG. 7, the covering 110 can include one or more layers 117a, 117b to achieve desired attributes for the covering 110. For example, the covering 110 can include a UVC blocking layer, a UVC reflective layer, and/or a thermal insulating layer. In one aspect, a single layer can be configured to block UVC, reflect UVC, and/or provide thermal insulation. In some examples, the covering 110 can include a base layer 117a and one or more secondary layers 117b. For instance, the covering 110 can include a base or foundation layer 117a configured to interface with the hand 101. In one aspect, the base layer 117a can be operable to block UVC light. In some examples, the base layer 117a can be a reflective layer operable to reflect UVC light toward the glove exterior 103. In some examples, the base layer 117a can be a thermally insulating layer configured to reduce heat transfer from the light source 120 to the glove interior 102. In one aspect, a secondary layer 117b disposed over the base layer 117a can include a reflective layer configured to reflect the UVC light toward the glove exterior 103. Although two layers are illustrated in FIG. 7, it should be recognized that the covering 110 can include any suitable number of layers in accordance with the principles disclosed herein. Non-limiting examples of suitable UV reflective material can include PTFE, LDPE, vinyl, polyester, metal film (e.g. Al, SS, etc), and the like.

In one aspect, the light source 120 and/or one or more of the optical fibers 121a-f can be removably coupled to the covering 110 (e.g., at 118 in FIG. 7), such as by at least one of a hook and loop fastener, a snap, a magnet, a button, or a clip. In one example, one or more of the optical fibers 121a-f can be associated or integrated with a textile 119, as described above. Other components of the germicidal glove 100, such as the light source 120, the power source 130, and/or the control system 150, can also be associated with the textile 119. Thus, the optical fibers 121a-f and any other component associated with the textile 119 can be removably secured to the covering 110 to facilitate removal and cleaning (e.g., washing) of the covering 110 separate from the electrical and optical components of the germicidal glove 100. The textile 119 can have any suitable configuration, such as an outer layer over some of the covering 110 (e.g., the palm portion 111, the finger portion 112a-d, the thumb portion 113, the wrist portion 114, and/or the back portion 115) or as an outer layer over substantially all of the covering 110.

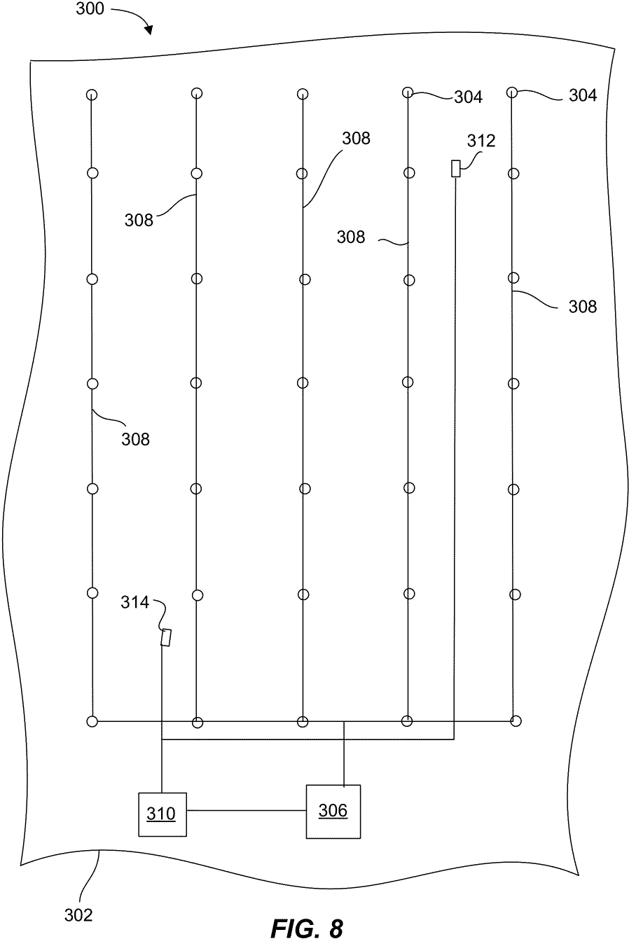

Although the above principles are exemplified via a glove, all of the above features can be fully integrated into a planar sheet (e.g. hand cloth or hand towel). FIG. 8 illustrates a germicidal hand cloth 300 formed as a planar sheet 302. LED lights 304 can be distributed about the planar sheet 302 and powered by a power source 306 via electrical lines 308. Operation of the lights can be selectively controlled by control system 310. For example, illumination parameters such as duration, frequency, intensity and/or illumination patterns can be varied based on user input or pre-programmed illumination profiles. In some cases, a user interface (not shown) can be used to allow a user to turn the device on/off, to select illumination parameters, and/or to select illumination profiles. Of course, optical fibers and one or more corresponding LED light sources can also be used (as described above in connection with FIGS. 1-7) in lieu of distributed LED lights directly mounted to the exterior surface (as described in FIG. 8).

Furthermore, optional sensors 312, 314 can be used to detect when the germicidal hand cloth is in contact with, or in sufficient proximity to, a surface to trigger illumination. Thus, the sensors (including 140, 141, 312, 314) can be pressure sensors, proximity sensors, induction sensors, or the like which can provide confirmation that a corresponding surface is within a treatment distance of the distributed light, i.e. emission point from the device.

The planar sheet 302 can generally be hand operative and have a size to permit handling with a single hand. As a general guideline, the planar sheet can have a minimum width and length of 8 cm and a maximum width and length of 60 cm, and in some cases from 10 cm to 30 cm width and length, or largest dimension if shaped as a non-quadrilateral. Accordingly, although the planar sheet can most often be rectangular or square in shape, other suitable shapes can include quadrilateral, circular, elliptical, polygonal, etc.

In another alternative, the germicidal hand cloth 300 can include at least one finger retention member on the interior surface and operable to receive a finger. For example, one or more finger loops or pockets can be attached to the interior surface to allow grasping and manipulation by a user. In still another alternative, one or both of the interior and exterior surface can include a rubberized coating to allow increased friction and grip.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A germicidal donnable hand cloth, comprising:
a fabric substrate having an interior operable to receive a hand and an exterior opposite the interior, and the fabric substrate being sized for use with a single hand; and
a light source supported by the fabric substrate, the light source being operable to emit ultraviolet C spectrum (UVC) light having a wavelength from about 200 nm to about 280 nm, wherein the UVC light is directed outwardly from the exterior to kill germs located remote from the fabric substrate.

2. The germicidal hand cloth of claim 1, wherein the fabric substrate is formed as a glove having a covering for a hand, the covering defining a glove interior operable to receive the hand, and a glove exterior opposite the glove interior.

3. The germicidal hand cloth of claim 1, wherein the hand cloth is formed as a planar sheet.

4. The germicidal hand cloth of claim 3, wherein the planar sheet has a minimum width and length of 8 cm and a maximum width and length of 60 cm.

5. The germicidal hand cloth of claim 3, wherein the interior includes at least one finger retention member operable to receive a finger.

6. The germicidal hand cloth of claim 1, wherein the UVC light has a wavelength from about 260 nm to about 270 nm.

7. The germicidal hand cloth of claim 1, wherein the light source comprises one or more light emitting diodes (LEDs).

8. The germicidal hand cloth of claim 1, wherein the fabric substrate comprises at least one of a base layer configured to interface with the hand, and a reflective layer over the base layer configured to reflect the UVC light toward the exterior.

9. The germicidal hand cloth of claim 8, wherein the base layer is operable to block the UVC light.

10. The germicidal hand cloth of claim 1, wherein the light source is removably coupled to the fabric substrate.

11. The germicidal hand cloth of claim 10, wherein the light source is removably coupled to the fabric substrate by at least one of a hook and loop fastener, a snap, a magnet, a button, or a clip.

12. The germicidal hand cloth of claim 2, wherein the covering comprises at least one of a palm portion, a finger portion, or a thumb portion, and the UVC light is directed from at least one of the palm portion, the finger portion, or the thumb portion.

13. The germicidal hand cloth of claim 12, further comprising one or more optical fibers operably coupled to the light source to distribute the UVC light from the at least one of the palm portion, the finger portion, or the thumb portion.

14. The germicidal hand cloth of claim 13, wherein the one or more optical fibers are integrated with a textile fabric.

15. The germicidal hand cloth of claim 13, wherein the one or more optical fibers are removably coupled to the covering.

16. The germicidal hand cloth of claim 15, wherein the one or more optical fibers are removably coupled to the covering by at least one of a hook and loop fastener, a snap, a magnet, a button, or a clip.

17. The germicidal hand cloth of claim 13, wherein the light source comprises one or more light sources located on the at least one of the palm portion, the finger portion, or the thumb portion to direct the UVC light from the at least one of the palm portion, the finger portion, or the thumb portion.

18. The germicidal hand cloth of claim 1, further comprising a power source supported by the fabric substrate and operably coupled to the light source.

19. The germicidal hand cloth of claim 18, wherein the fabric substrate comprises a pocket operable to receive the power source.

20. The germicidal hand cloth of claim 1, further comprising a sensor supported by the fabric substrate, the sensor being operable to detect contact on the exterior, wherein when contact is detected the light source emits the UVC light.

21. The germicidal hand cloth of claim 20, wherein the UVC light is directed toward the exterior from a plurality of locations about the fabric substrate, and the sensor comprises a plurality of sensors, each sensor associated with one or more of the plurality of locations about the fabric substrate, and wherein when contact is detected by one of the plurality of sensors, the light source emits the UVC light from the one or more of the plurality of locations associated with the one of the plurality of sensors.

22. The germicidal hand cloth of claim 1, further comprising a thermally insulating layer configured to reduce heat transfer from the light source to the interior.
